(19) United States
(12) Patent Application Publication
(10) Pub. No.: US 2015/0040907 A1
(43) Pub. Date: Feb. 12, 2015

(54) VALVED BREATHING DEVICE PROVIDING ADJUSTABLE EXPIRATION RESISTANCE FOR THE TREATMENT OF SLEEP DISORDERED BREATHING

(71) Applicants: SAL T. HAKIM, PHOENIX, AZ (US); JOSHUA J. HAKIM, PHOENIX, AZ (US)

(72) Inventors: SAL T. HAKIM, PHOENIX, AZ (US); JOSHUA J. HAKIM, PHOENIX, AZ (US)

(21) Appl. No.: 14/254,716

(22) Filed: Apr. 16, 2014

Related U.S. Application Data
(60) Provisional application No. 61/863,101, filed on Aug. 7, 2013.

(51) Int. Cl. A61M 16/20; A61M 16/06

(52) U.S. Cl. CPC A61M 16/201; A61M 16/0666; A61M 16/0622
USPC 128/205.24

(57) ABSTRACT

Some embodiments of the present disclosure include nasal pillows and a valve assembly for the treatment of sleep disordered breathing. The valve assembly may include a retainer having retainer slots attached to a valve body, a flexible membrane positioned between the valve body and the retainer, the flexible membrane configured to block the retainer slots when a user exhales, and a mask coupling configured to engage with a nasal mask or a nasal pillow support. The valve body may include at least one expiratory port and a mechanism for adjusting a size of the expiratory port, wherein when the size of the expiratory port is decreased, air resistance increases, and when the size of the expiratory port is increased, air resistance decreases. In embodiments, the adjustment mechanism may be a sleeve or an adjustment screw and the nasal pillows may be replaced with a nasal mask.

### RELATED APPLICATION

This application claims priority to provisional patent application U.S. Ser. No. 61/863,101 filed on Aug. 7, 2013, the entire contents of which is herein incorporated by reference.

### BACKGROUND

The embodiments herein relate generally to breathing devices, and more particularly, to a valve providing adjustable expiration resistance for the treatment of sleep disordered breathing.

Sleep disordered breathing includes obstructive sleep apnea (OSA) and snoring. OSA is a medical condition associated with several symptoms and morbidities, including snoring. OSA occurs during sleep because the muscles of the tongue and surrounding tissue in the upper airways (UAs) relax, causing them to shift toward the back of the mouth with crowding and/or obstruction of airflow. When obstruction is complete, breathing stops (apnea), leading to a decline in oxygen levels in the blood. Hypopnea is when airflow is reduced. When obstruction is incomplete and air flows through the narrowed UA, air moves faster leading to the vibration of tissues and snoring noises.

Conventionally, continuous positive airway pressure (CPAP) therapy is the preferred treatment for OSA. CPAP increases pressure in the UAs (oropharyngeal region), which acts as a stent, to reduce crowding and prevent obstruction, thus allowing air to continue to flow in the UAs. CPAP also increases lung volume, making the UAs less susceptible to collapse. The combination of the increase in lung volume and the increase in airway pressure acts together to prevent airway collapse during sleep, thus preventing OSA and snoring.

Expiratory positive airway pressure (EPAP) therapy is a more recent treatment for OSA. EPAP increases lung volume and provides a positive pressure during expiration, particularly at the end of expiration when UAs are most susceptible to collapse and, thus, EPAP is also useful in treating OSA and snoring. The difference between CPAP and EPAP is that, in CPAP, pressure is positive in both expiration and inspiration, whereas in EPAP, pressure is positive only during expiration. Lung volume changes are comparable with EPAP and CPAP.

CPAP typically requires a user to wear a mask that is connected, or tethered via a large tube, to a CPAP machine, limiting the movement and positions in which a user may sleep. Although EPAP has been known for many years to be effective in the treatment of sleep disordered breathing, only recently have EPAP devices been introduced into the market as a new option for treating sleep disordered breathing. Current EPAP devices include disposable nasal plugs inserted inside the nostrils and affixed to a user's nose with an adhesive. EPAP devices allow air to flow in with ease during inspiration, but create resistance to airflow during expiration, creating positive pressure during the entire phase of expiration. While EPAP devices that are currently on the market do not require a user to be tethered to a machine, the amount of resistance provided by EPAP devices is substantially fixed and may cause hypoventilation and carbon dioxide retention. One resistance is unlikely to be equally suitable for all patients because of the differences in body size and lung volume. Moreover, current EPAP devices are uncomfortable and inconvenient to use.

Therefore, what is needed is an OSA and snoring treatment device that is comfortable and convenient to use and does not require a user to be tethered to a machine, while also providing for variable resistance, which allows air to flow out without compromising ventilation and without significant carbon dioxide retention.

### SUMMARY

Some embodiments of the present disclosure include a valve assembly attached to nasal pillows for the treatment of sleep disordered breathing. The valve assembly may include a retainer having retainer slots, the retainer attached to a valve body, a flexible membrane positioned between the valve body and the retainer. The flexible membrane may be configured to block the retainer slots when a user exhales, and a mask coupling may be configured to engage with a nasal mask or a nasal pillow support. The valve body may include at least one expiratory port and a mechanism for adjusting a size of the at least one expiratory port, wherein when the size of the at least one expiratory port is decreased, air resistance increases and pressure during expiration rises, and when the size of the at least one expiratory port is increased, air resistance decreases. In some embodiments the adjustment mechanism may be a sleeve or an adjustment screw.

### BRIEF DESCRIPTION OF THE FIGURES

The detailed description of some embodiments of the invention is made below with reference to the accompanying figures, wherein like numerals represent corresponding parts of the figures.

FIG. 1 is a perspective view of one embodiment of the present invention shown in use.

FIG. 2 is a perspective view of one embodiment of the present invention.

FIG. 3 is an exploded view of the valve parts in one embodiment of the present invention.

FIG. 4 is a perspective view of the assembled valve parts in one embodiment of the present invention.

FIG. 5 is a section view of one embodiment of the valve used in the present invention, taken along line 5-5 in FIG. 2.

FIG. 6 is a front perspective view of one embodiment of the present invention.

FIG. 7 is a rear perspective view of one embodiment of the present invention.

FIG. 8 is a side view of one embodiment of the present invention.

FIG. 9 is a perspective view of one embodiment of the present invention showing an alternate mechanism for holding the pillows in place.

FIG. 10 is a perspective view of one embodiment of the valve used in the present invention.

### DETAILED DESCRIPTION OF CERTAIN EMBODIMENTS

In the following detailed description of the invention, numerous details, examples, and embodiments of the invention are described. However, it will be clear and apparent to one skilled in the art that the invention is not limited to the embodiments set forth and that the invention can be adapted for any of several applications.

The device of the present disclosure may be used to treat sleep disordered breathing and may comprise the following elements. This list of possible constituent elements is intended to be exemplary only, and it is not intended that this list be used to limit the device of the present application to just these elements. Persons having ordinary skill in the art relevant to the present disclosure may understand there to be equivalent elements that may be substituted within the present disclosure without changing the essential function or operation of the device.

1. Valve Providing Adjustable Expiration Resistance

2. Nasal Attachment

3. Mechanism to Hold Nasal Attachment in Place

The various elements of the valve providing adjustable expiration resistance for the treatment of sleep disordered breathing of the present disclosure may be related in the following exemplary fashion. It is not intended to limit the scope or nature of the relationships between the various elements and the following examples are presented as illustrative examples only.

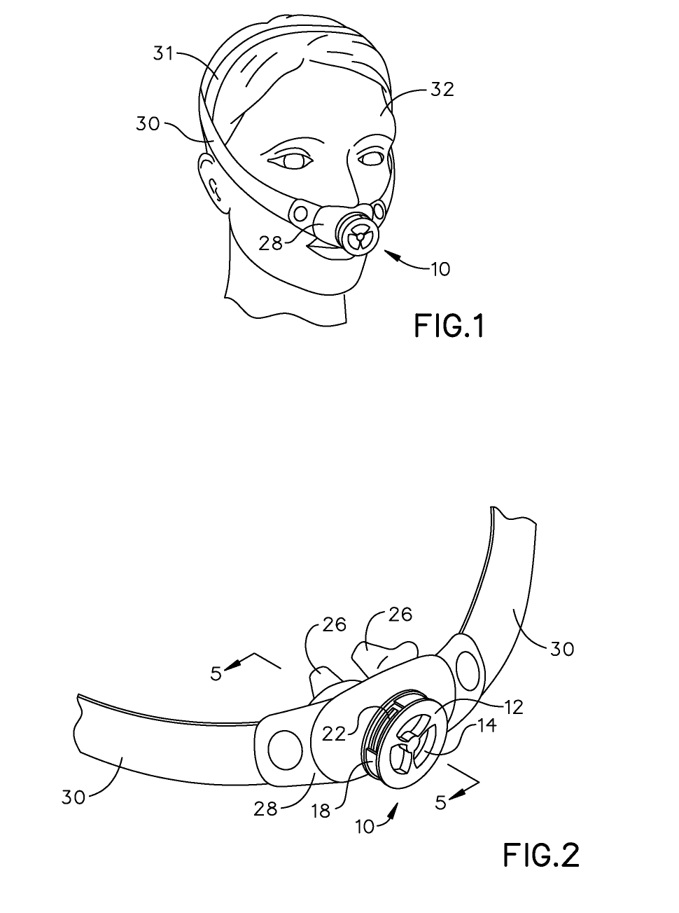

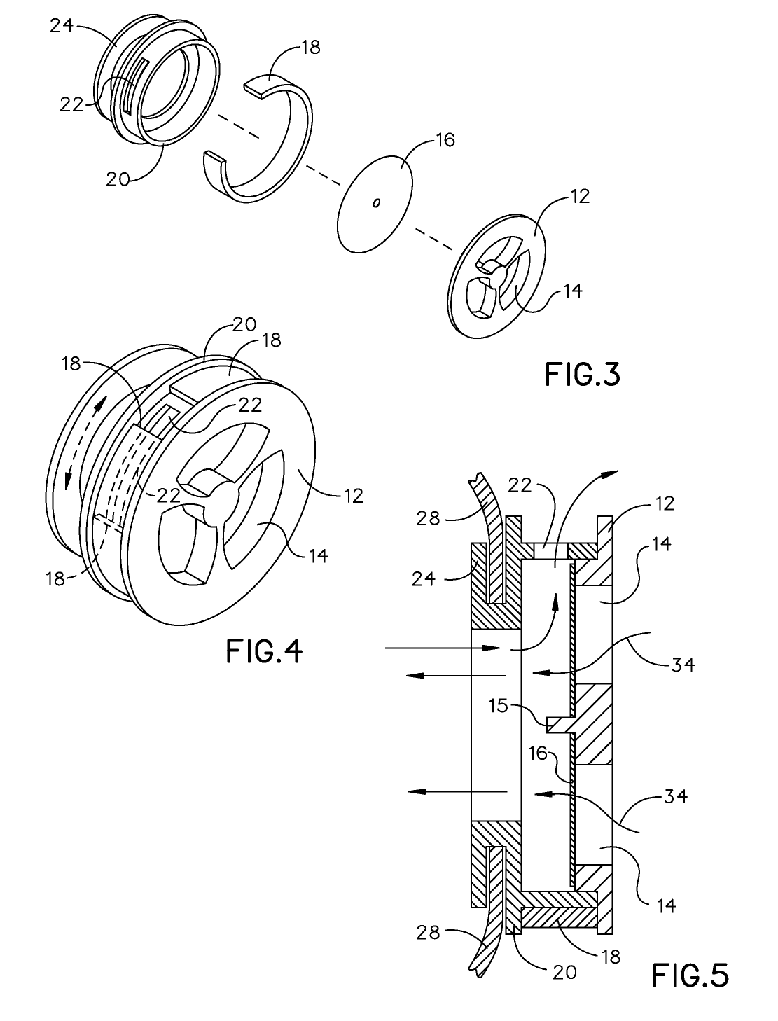

By way of example, and referring to FIGS. 1-10, some embodiments of the system for treating sleep disordered breathing of the present disclosure comprise a valve assembly 10 configured to provide adjustable expiration resistance to a user 32. As shown in FIGS. 3-5, the valve assembly 10 may be substantially cylindrical and may comprise a retainer 12 attached to a valve body 20, wherein a flexible membrane 16 is positioned between the valve body 20 and the retainer 12. The retainer 12 may have a retainer post 15 and a plurality of retainer slots 14, such that when a user 32 inhales, inhaled air 34 flows through the retainer slots 14, pushing the flexible membrane 16 toward the user 32 and allowing the inhaled air 34 to continue to flow through the valve assembly 10 to the user 32 with minimal resistance.

The valve body 20 may include an expiratory port 22, through which exhaled air is configured to flow. In some embodiments, the valve body may 20 may include a plurality of expiratory ports 22. A rotatable sleeve 18 may partially encircle the valve body, as shown in FIGS. 3 and 4, the sleeve 18 configured to adjustably cover any amount of the expiratory port 22, such as none of the expiratory port or any other percentage of the expiratory port 22, such as from about 0% to about 100% of the expiratory port 22, to increase or decrease expiratory resistance to the exhaled air. For a greater resistance or a greater pressure, the user 32 would cover a greater percentage of the expiratory port 22 with the sleeve 18 or, for less resistance, the user 32 would cover a lesser percentage of the expiratory port 22 with the sleeve 18. When a user 32 exhales, the flexible membrane 16 may block air from exiting through the retainer slots 14, such that exhaled air may only exit the valve assembly 10 through the expiratory port 22.

Alternatively, as shown in FIG. 10, an alternate valve assembly 52 may comprise an alternate valve body 54, the alternate valve body 54 comprising a plurality of valve outer body holes 58 and an adjustment screw 56 on the front of the valve body 54 having adjustment screw holes 60, wherein the amount of resistance provided to the exhaled air is adjusted by twisting the adjustment screw 56.

In even further embodiments, the mechanism for adjusting the resistance of the exhaled air may comprise a slider with a plurality, such as three or four, discrete, different sized holes, wherein the slider allows exhaled air to flow through the desired fixed holes.

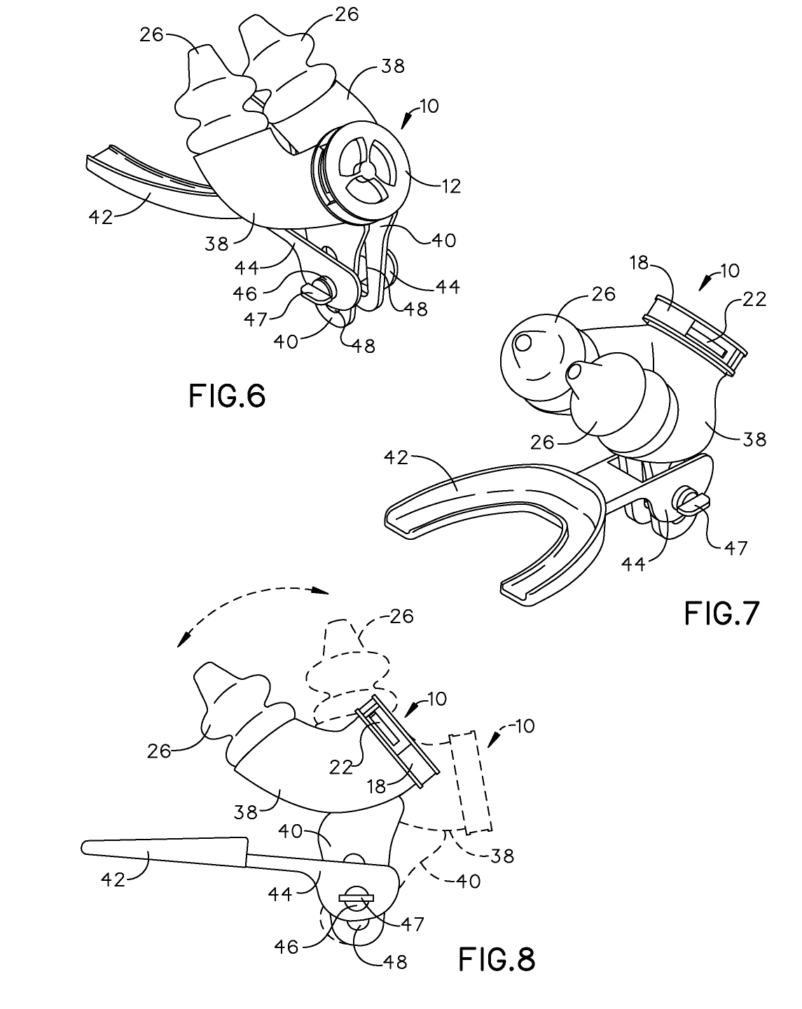

In either embodiment, the valve assembly 10, 52 may comprise a mask coupling 24, 62 configured to engage with a mask 28, as shown, for example, in FIGS. 1, 2, 5, and 9, or with a pillow support 38, as shown in FIGS. 6-8. The mask coupling 24, 62 on the valve assembly 10, 52 may vary depending on the design of the attachment point with which it will engage.

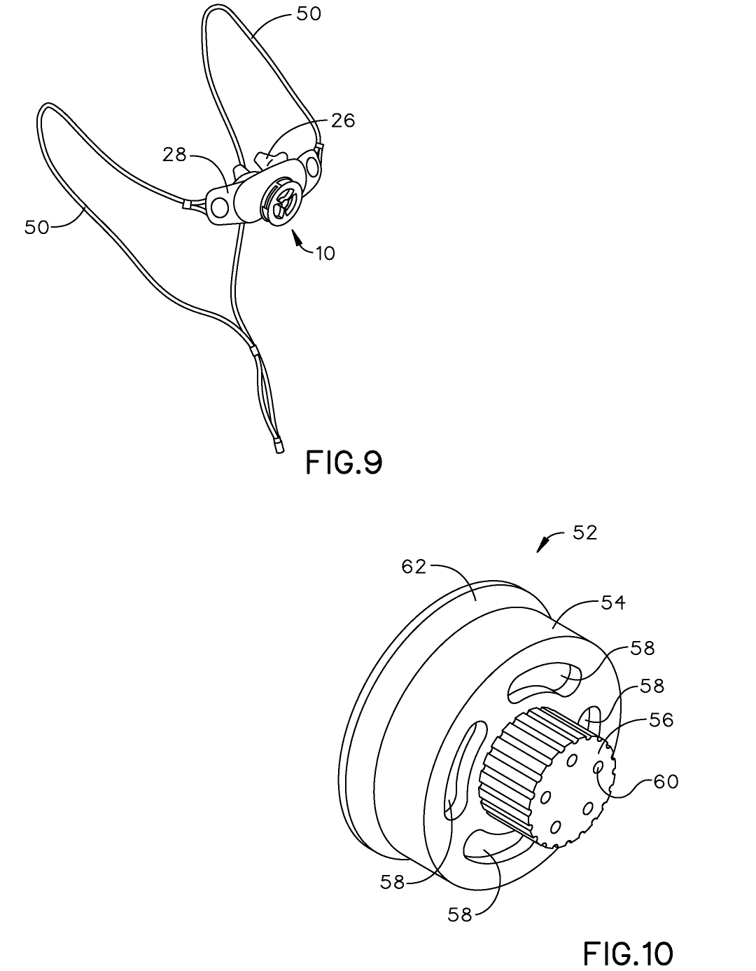

In some embodiments, the mask 28 may comprise a plurality of nasal pillows 26, each of which include an air passageway for inhalation and exhalation, the nasal pillows 26 configured to engage with a user's nostrils, such that when a user 32 inhales or exhales through their nostrils, the inhaled or exhaled air passes through the valve assembly 10, 52. Alternatively, the nasal pillows 26 may be replaced by another type of nasal mask, such as a mask that fits around the nose instead of fitting against the nostrils. The mask 28 may be configured to attach to a user using any suitable means, such as by being attached to a head band 30 and a head band top strap 31, each configured to wrap around a user's head, as shown in FIG. 1, or by being attached to ear straps 50 that are configured to wrap around a user's ears, as shown in FIG. 9. However, any other suitable means may be used to secure the mask to a user.

As discussed above, in embodiments the valve assembly 10, 52 may be attached to a rigid pillow support 38 having two holes to hold the soft, removable nasal pillows 26, as shown in FIGS. 6-8. A pair of removable, nasal pillows 26, which may be configured to externally engage with a user's nostrils, may extend upwardly from the pillow support 38, while support arm 40 may extend downwardly from the pillow support 38. The support arm 40 may be configured to attach to a mouthpiece 42. For example, in some embodiments, the support arm 40 may comprise an adjustment slot 48 and the mouthpiece 42 may comprise a mouthpiece arm 44 having an adjustment knob 47, wherein a pivot rod 46 is attached to the adjustment knob 47 and extends through the adjustment slot 48. The adjustment knob 47 may be configured to adjust an angle of the mouthpiece 42 relative to the pillow support 38, as shown in FIG. 8, such that the valve assembly 10, 52 may accommodate a variety of face shapes and sizes.

To use the valve assemblies of the present disclosure, a user may insert the nasal pillows into the nasal cavities and secure the valve assembly in place. When a user inhales, the valve assembly may provide little to no resistance; however, when a user exhales, the valve assembly may provide the desired level of resistance. A user may set the resistance to the desired level by either rotating the sleeve to cover more or less of the expiratory port, or by rotating the adjustment screw. As a result of the resistance on the exhaled air, sleep disordered breathing may be treated, and the valve assembly may help prevent OSA and snoring.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A valve assembly for the treatment of sleep disordered breathing, the valve assembly comprising:
a valve body comprising at least one expiratory port; and
a mechanism for adjusting a size of the at least one expiratory port through which exhaled air may flow,
wherein:
when the size of the at least one expiratory port through which exhaled air may flow is decreased, air resistance increases; and
when the size of the at least one expiratory port through which exhaled air may flow is increased, air resistance decreases.

2. The valve assembly of claim 1, further comprising a rotatable sleeve partially encircling the valve body, wherein:
the valve body is substantially cylindrical and the expiratory port is located on a side surface of the valve body; and
the rotatable sleeve is configured to rotate to cover from about 0 to about 100% of the expiratory port.

3. The valve assembly of claim 1, wherein the valve body further comprises an adjustment screw configured to control an percentage of the at least one expiratory port that is open and not blocked.

4. The valve assembly of claim 1, further comprising:
a retainer comprising a plurality of retainer slots on a surface thereof, the retainer being attached to the valve body; and
a flexible membrane positioned between the valve body and the retainer, the flexible membrane being configured to block exhaled air from exiting the valve assembly through the retainer slots in the retainer.

5. The valve assembly of claim 1, wherein the valve body comprises a mask coupling configured to engage with a member selected from the group consisting of a nasal mask and a pillow support.

6. A system for the treatment of sleep disordered breathing, the system comprising:
an attachment configured to engage with a user's nasal passages, wherein the attachment is a member selected from the group consisting of a plurality of nasal pillows and a nasal mask that fits around the user's nose;
a valve assembly attached to the plurality of nasal pillows, the valve assembly comprising:
a valve body comprising at least one expiratory port; and
a mechanism for adjusting a size of the at least one expiratory port through which exhaled air may flow,
wherein:
when the size of the at least one expiratory port through which exhaled air may flow is decreased, air resistance increases; and
when the size of the at least one expiratory port through which exhaled air may flow is increased, air resistance decreases.

7. The system of claim 6, wherein:
the valve body further comprises:
a retainer comprising a plurality of retainer slots on a surface thereof, the retainer being attached to the valve body;
a flexible membrane positioned between the valve body and the retainer, the flexible membrane being configured to block exhaled air from exiting the valve assembly through the retainer slots in the retainer; and
the valve assembly further comprises a rotatable sleeve partially encircling the valve body, wherein the valve body is substantially cylindrical and the expiratory port is located on a side surface of the valve body, and the rotatable sleeve is configured to rotate to cover from about 0 to about 100% of the expiratory port.

8. The system of claim 6, wherein the valve body further comprises a mask coupling configured to engage with a member selected from the group consisting of a nasal mask and a pillow support.

9. A method of treating sleep disordered breathing in a user, the method comprising:
securing a valve assembly to a user's nostrils; and
adjusting resistance provided by the valve assembly on exhaled air,
wherein:
the valve assembly comprises:
a valve body comprising at least one expiratory port;
a mechanism for adjusting a size of the at least one expiratory port through which exhaled air may flow; and
a mask coupling configured to engage with at least one member selected from the group consisting of a nasal mask comprising a pair of nasal pillows, a support pillow attached to a pair of nasal pillows, and a nasal mask configured to fit around a user's nose,
when the size of the at least one expiratory port through which exhaled air may flow is decreased, air resistance increases; and
when the size of the at least one expiratory port through which exhaled air may flow is increased, air resistance decreases.

10. The method of claim 10, wherein:
the valve body further comprises:
a retainer comprising a plurality of retainer slots on a surface thereof, the retainer being attached to the valve body;
a flexible membrane positioned between the valve body and the retainer, the flexible membrane being configured to block exhaled air from exiting the valve assembly through the retainer slots in the retainer; and
the valve assembly further comprises a rotatable sleeve partially encircling the valve body, wherein the valve body is substantially cylindrical and the expiratory port is located on a side surface of the valve body, and the rotatable sleeve is configured to rotate to cover from about 0 to about 100% of the expiratory port.
